(19) United States
(12) Patent Application Publication
(10) Pub. No.: US 2025/0362243 A1
(43) Pub. Date: Nov. 27, 2025

(54) Soil Moisture Sensor

(71) Applicant: Wisconsin Alumni Research Foundation, Madison, WI (US)

(72) Inventors: Bhuvana Krishnaswamy, Madison, WI (US); Yoganand Biradavolu, Madison, WI (US)

(21) Appl. No.: 18/670,878

(22) Filed: May 22, 2024

(51) Int. Cl. G01N 22/04; G01N 33/24; H02J 50/00

(52) U.S. Cl. CPC G01N 22/04; G01N 33/246; H02J 50/001

(57) ABSTRACT

A soil moisture sensor employs a buried transponder that harvests energy from a surface transmitter to provide a second transmission indicating the received radio power by the transponder being a function of soil moisture.

### STATEMENT REGARDING FEDERALLY SPONSORED RESEARCH OR DEVELOPMENT

### CROSS REFERENCE TO RELATED APPLICATION

### BACKGROUND OF THE INVENTION

The present invention relates to a method and apparatus for measuring soil moisture and, in particular, to a soil moisture sensor deducing moisture from radiofrequency signal strength measured at a buried transponder.

Low-cost, in-situ soil moisture measurements in agricultural fields spanning acres of land are important in making informed irrigation decisions and protecting groundwater. Conventionally, soil moisture is measured using soil contacting electrodes that can detect current flow through the soil or soil capacitance reflecting moisture contact. Such contacting electrodes are subject to being fouled and may provide overly localized measurements. For this reason, noncontact sensing, for example, using ground penetrating radar, may be considered as an alternative.

The costs associated with radar-type systems requiring specialized and expensive electronics has suggested the use of passive, radiofrequency identification (RFID) tags buried in the earth to sense moisture. Such tags respond to an external radiofrequency signal (for example, from the surface) to communicate codes through backscatter of the external radiofrequency signal back to the surface. The strength and other characteristics of the backscattered signal provide a coarse measure of soil moisture.

### SUMMARY OF THE INVENTION

The present invention provides a soil moisture measurement using a contactless radiofrequency attenuation measurement. In contrast to RFID tags, the invention determines soil moisture through attenuation of a radio signal from the surface to a buried sensor during an energy harvesting process. This energy harvesting powers a return signal encoding the received energy value (most simply by the time delay of the reply signal). By eliminating the reliance on backscatter, improved signal strength (and potentially improved resolution and/or depth of measurement) is obtained while eliminating the need for the surface unit to have sophisticated radio signal characterization circuitry.

In one specific embodiment, the invention provides a radio transceiver for transmitting a first radio signal and receiving a second signal, the radio transceiver adapted to be moved above the surface of the soil among locations. A transponder circuit is adapted for burying in the soil at a predetermined depth and operates to receive and store energy from the first radio signal and use the stored energy to transmit the second radio signal and to encode the second radio signal with an encoding indicating a strength of the first radio signal. An output circuit communicating with the radio transceiver provides an output indicating soil moisture as a function of the strength of the encoding of the second radio signal.

It is thus a feature of at least one embodiment of the invention to provide improved measurement of soil moisture by capturing transmission energy strength at the transponder and employing energy harvesting to provide a signal relaying this information having improved signal strength and measurement resolution.

The encoding may be a delay time between the reception of the first radio signal and transmission of the second radio signal.

It is thus a feature of at least one embodiment of the invention to provide a simple encoding that does not require precise timing at the surface transponder as might be required, for example, for radar-type systems monitoring transmission delay.

In some embodiments, the encoding provides a range of transmission delays more than 1 microsecond for soil moisture between 20% and 80%.

It is thus a feature of at least one embodiment of the invention to provide a mapping between transmission delay and soil moisture that can provide improved resolution of soil moisture with lower precision timing circuits, for example, than those based on the clock in a microprocessor.

The transponder circuit may transmit the second radio signal with a second encoding independent of the first encoding.

It is thus a feature of at least one embodiment of the invention to provide extra data, for example, that may distinguish between transponders or convey additional information about the soil.

The soil moisture sensor may further include an electrically insulating stake having the transponder attached thereto and adapted to bury the transponder at the predetermined depth by insertion of the stake into the soil and to provide a surface visible portion.

It is thus a feature of at least one embodiment of the invention to provide a simple method of precisely installing the transponders at a predetermined depth allowing ready location of the transponders when measurement is required.

The soil moisture sensor may further include a second transponder circuit adapted for positioning at a surface of the soil, and the output circuit may combine the second radio signal and third radio signal to extract a net attenuation of the first radio signal by the soil.

It is thus a feature of at least one embodiment of the invention to compensate for variations in the position of the transmitter antenna above the soil and/or electrical properties of the air which have been determined by the inventors to change significantly, for example, depending on humidity.

The second transponder circuit may transmit the second radio signal with a second encoding independent of the first encoding and uniquely identifying the second transponder circuit.

It is thus a feature of at least one embodiment of the invention to provide a separate encoded data from the second transponder either to help distinguish it from other transponders on the stake or from multiple transponders at different stakes distributed throughout an agricultural field.

The soil moisture sensor may further include an input circuit for receiving a soil type and the output circuit may employ the soil type and the encoding indicating a strength to provide the output indicating the soil moisture as a function of the strength of the encoding of the second radio signal and the soil type.

It is thus a feature of at least one embodiment of the invention to provide improved accuracy for different soil types.

These particular objects and advantages may apply to only some embodiments falling within the claims and thus do not define the scope of the invention.

### BRIEF DESCRIPTION OF THE DRAWINGS

FIG. 1 a schematic representation of a soil measurement system according to the present invention providing a transmitter movable over the surface of the soil with respect to multiple transponders supported on stakes inserted into the ground, with an inset showing a transponder block diagram.

FIG. 2 is a system block diagram showing the processing of multiple inputs to determine and display soil moisture.

FIG. 3 is a timing diagram showing a mutually aligned energizing signal from the surface transmitter, energy accumulation by a transponder triggering a reply signal by that transponder, and the reply signal showing encoding by the delay time of the transmission as well as a bit pattern encoded in the reply signal; and

FIG. 4 is an elevational cross-sectional view of a stake holding a transponder in the soil as may be interrogated by a drone system for providing the movable surface transmitter of FIG. 1.

### DETAILED DESCRIPTION OF THE PREFERRED EMBODIMENT

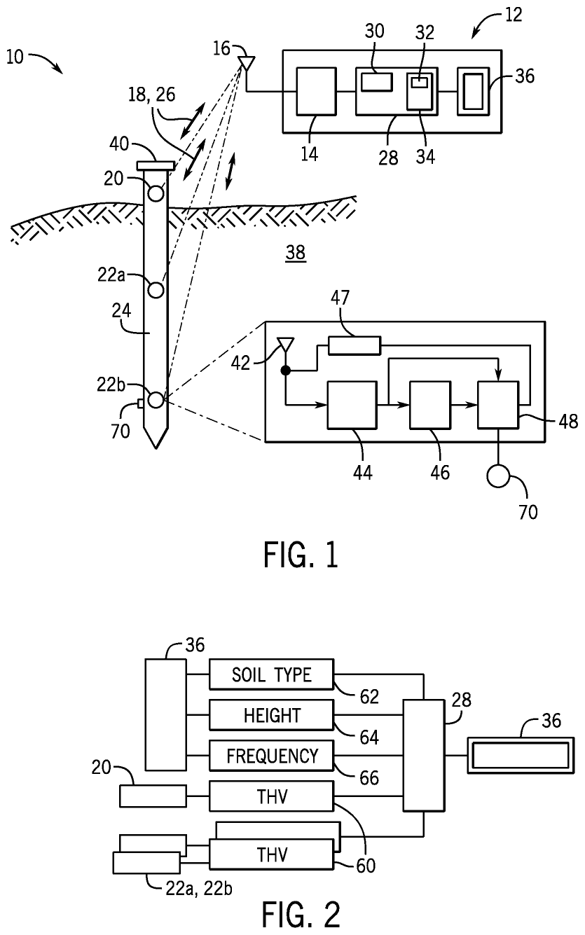

Referring now to FIG. 1, a soil moisture sensing system 10, in one illustrative embodiment of the present invention, may provide for a surface transmitter unit 12 providing a radio transceiver 14 communicating with an antenna 16 for transmitting a radio-frequency, energizing signal 18 to a surface transponder 20 and one or more buried transponders 22a and 22b, for example, supported on a stake 24. As will be described in more detail below the transponders 20, 22a, and 22b provide radio reply signals 26 also received by antenna 16 to be processed by the transceiver 14.

The surface transmitter unit 12 may be moved freely over the surface of the soil 38 in which the transponders 20, 22a and 22b are fixed and buried. In one example, the surface transmitter unit 12 may be a handheld device that may be carried around the agricultural field for measurement and operated by battery power.

The transceiver 14 of the surface transmitter unit 12 may communicate with a controller 28 having one or more processors 30 and executing a stored program 32 held in computer memory 34. The controller 28 may communicate with a user interface 36, for example, providing a graphic screen and keyboard or the like for outputting and receiving inputs to and from a user. Generally, the surface transmitter unit 12 will be battery powered to be fully portable to be moved among different stakes 24.

The stake 24 may be constructed of a polymer material to resist the elements and reduce radiofrequency attenuation or reflection. Desirably the stake 24 is of sufficient strength for insertion into the soil 38 so that the surface transponder 20 is at the surface of the soil 38 and exposed thereat and the buried transponders 22a and 22b are at predetermined depths, for example, selected from 5, 10 and 15 cm below the surface of the soil 38. An upper surface of the stake 24 may protrude and may provide for a visual or similar marker 40 to help a user or drone system to locate the stake 24.

Each surface transponder 20 and buried transponder 22 maybe fully encapsulated for protection against moisture and the soil and thus need not have galvanic connections thereto. Each surface transponder 20 and buried to transponder 22 may provide an antenna 42 receiving the energizing signal 18 and providing this received signal to an energy harvester 44. The energy harvester 44 may include an antenna impedance matching system and a set of rectifiers for charging an associated capacitance storing electrical energy derived from the energizing signal 18. In one nonlimiting example, the energy harvester 44 may be a twelve-stage Dixon charge pump using Schottky diodes and providing 100 μF of capacitive electrical energy storage.

The voltage on the capacitance of the energy harvester 44 may be provided to a threshold detector 46, for example, a comparator, providing an output signal when a given threshold of energy has been stored in the capacitance and providing this output signal to controller 48. Controller 48 may be a low-power microprocessor or the like, and the energy harvester 44 further provides electrical power to the controller 48 to move it to a wake state. For this latter purpose, the energy harvester 44 may incorporate a voltage regulator or other power management components.

The controller 48 upon receiving the signal from the energy harvester indicating that a predetermined and fixed level of energy has been stored, triggers a reply signal 26 through a transmitter 47, preferably operating at a different frequency than the energizing signal 18. The reply signal 26 is provided to the antenna 42 to be received by the surface transmitter unit 12 for processing as will be described.

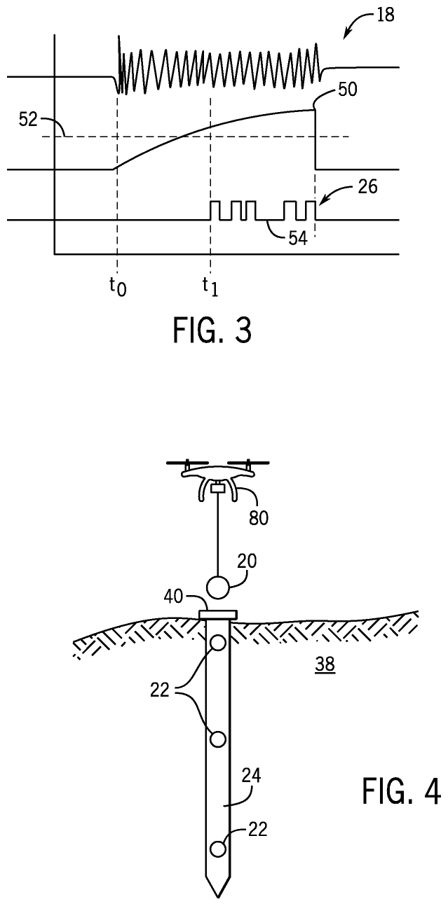

Referring momentarily to FIG. 3, soil moisture measurements are made initiating the transmission of the energizing signal 18 at time to by the surface transmitter unit 12. This energizing signal 18, for example, may be at 902 MHz carrier frequency, for example, modulated by a continuous 1 kHz sine wave and activated periodically only for a predetermined duration sufficient to fully energize buried transponders 22 at a maximum intended depth. At the time of initiation of the energizing signal 18, an internal clock is initiated by the controller 28.

The energizing signal 18, as received at the transponders 20, 20a and 20b, charges the capacitance of the energy harvester 44 as indicated by charge level 50 until it reaches a predetermined threshold value 52 at which time t₁ a signal is provided to the controller 48 causing it to send out the reply signal 26 powered by the capacitive energy of the energy harvester 44. The capacitance of the energy harvester 44 and the threshold value 52 are selected so that for a range of soil moisture, for example, between 20% and 80%, differences in delay time between t₀ and t₁ will be over 1 μs and typically over 1 ms allowing this time to be precisely measured with simple circuitry in the surface transmitter unit 12 at high resolution. Resolutions in received energy as small as one dBm may be obtained using a processor clock as a time base.

The reply signal 26 thus encodes the strength of the received energizing signal 18 by the time delay between t₀ and t₁ which will be a function of the received power of the energizing signal 18. The reply signal 26 may also provide an embedded bit pattern 54, for example, by modulation of the reply signal 26. The embedded bit pattern 54 may uniquely identify the surface transponder 20 or buried transponder 22a and 22b and/or may encode additional information as will be discussed below. In some embodiments, this bit pattern 54 may be also or alternatively used to communicate received energy strength.

The reply signal 26 will generally have a different frequency than the energizing signal 18 and may, for example, be at 915 MHz and may encode the bit pattern 54 in a 16-bit frequency shift modulated encoding process at a bit rate of 204 kbps. At the conclusion of this reply signal 26 and energizing signal 18, t₃, the capacitance of the energy harvester 44 may be discharged either through the energy consumed in the transmission and in operation of the controller 48 (estimated to be approximately 22 micro Joules) or by a shorting of that capacitance to be ready for another transmission of energizing signal 18.

Referring now also to FIG. 2, the controller 28 may receive signals from each of the transponders 20 and 22 at associated times t₁ to determine transmission hold off (THV) values 60 for each transponder 20 and 22. These hold off times, derived from a charging time of the capacitance of the energy harvester 44, will generally be a logarithmic function of the received energy which may be corrected at the surface transmitter unit 12.

The soil moisture sensing system 10, may receive, for example, as communicated to the surface transmitter unit 12 through the interface 36, any of the following additional information, including: an indication of soil type 62, an estimated height 64 of the transmitter antenna 16 above the ground, and a frequency of the first transmission 66. This additional information may then be used to calculate an output, for example, displayed at the interface 36 indicating soil moisture at one or more depths of the buried transponders 20.

Generally, this calculation of soil moisture may be performed either by a model, for example, using Frii's transmission equation describing the propagation of radio waves and Topp's equation providing a function of soil relative permittivity as related to soil moisture per G C Topp, M Yanuka, W D Zebchuk, and S Zegelin, Determination of electrical conductivity using time domain reflectometry: Soil and water experiments in coaxial lines, Water Resources Research, 24(7):945-952, 1988 hereby incorporated by reference. Alternatively or in addition this relationship may be established empirically with the results stored in a lookup table or the like. The transmission loss to the surface transponder 20 may be determined first and used to adjust the computed values of transmission loss to the buried transponders 22a and 22b to approximate the transmission loss that would be measured if the antenna 16 were at the location of the surface transponder 20.

Ideally the antenna 16 is located directly above the stake 24 in alignment with the transponders 20a, 22 and 22b, otherwise angular offsets must be accommodated such as may be done trigonometrically.

It will be appreciated that the controller 28 may further receive additional inputs such as soil temperature, for example, measured by a soil temperature sensor 70 (shown in FIG. 1) whose value may be communicated through the bit pattern 54 shown in FIG. 3. In this case the various transponders 20, 22a and 22b may be distinguished simply by their order of response (expected to be shortest for surface transponder 20 and then progressively longer for buried transponders 22a and 22b) or through the use of a longer bit pattern 54 that can accommodate both types of data.

Referring now to FIG. 4 it will be appreciated that in one embodiment the surface transponder 20 need not be incorporated into the stake 24 but may be moved among stakes 24 for additional cost savings. In one embodiment, a drone 80 incorporating the surface transmitter unit 12, may fly over the agricultural field to position the surface transponder 20 at each stake 24 to make the necessary soil moisture measurements. A telemetry output may be provided in lieu of interface 36, with the drone carrying the surface transponder 20 and the surface transmitter unit.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A soil moisture sensor comprising:
a radio transceiver for transmitting a first radio signal and receiving a second signal, the radio transceiver adapted to be moved above a surface of soil among locations;
a transponder circuit adapted for burying in the soil at a predetermined depth and operating to receive and store energy from the first radio signal and to use the stored energy to transmit the second radio signal and to encode the second radio signal with an encoding indicating a strength of the first radio signal; and
an output circuit communicating with the radio transceiver providing an output indicating soil moisture as a function of the strength of the encoding of the second radio signal.

2. The soil moisture sensor of claim 1 wherein the encoding is a delay time between reception of the first radio signal and transmission of the second radio signal.

3. The soil moisture sensor of claim 2 wherein the encoding provides a range of transmission delays of more than 1 ms for soil moisture between 20% and 80%.

4. The soil moisture sensor of claim 2 wherein the transponder circuit further transmits the second radio signal with a second encoding independent of the first encoding.

5. The soil moisture sensor of claim 1 wherein the transponder harvests energy from the first radio signal by rectification of the first radio signal and storage in a capacitance.

6. The soil moisture sensor of claim 1 further including an electrically insulating stake having the transponder attached thereto and adapted to bury the transponder at the predetermined depth by insertion of the stake into the soil and to provide a surface visible portion.

7. The soil moisture sensor of claim 1 further including a second transponder circuit adapted for positioning at a surface of the soil and operating to receive and store energy from the first radio signal and to use the stored energy to transmit a third radio signal with an encoding indicating a strength of the first radio signal; and
wherein an output circuit combines the second radio signal and third radio signal to extract a net attenuation of the first radio signal by the soil.

8. The soil moisture sensor of claim 7 wherein the encoding of the second transponder is a delay time between reception of the first radio signal and transmission of the third radio signal.

9. The soil moisture sensor of claim 8 wherein the second transponder circuit further transmits the second radio signal with a second encoding independent of the first encoding and uniquely identifying the second transponder circuit.

10. The soil moisture sensor of claim 7 further including an electrically insulating stake having the transponder and second transponder attached thereto and adapted to bury the transponder at the predetermined depth by insertion of the stake into the soil when the second transponder is at the surface of the soil.

11. The soil moisture sensor of claim 1 further including an input circuit for receiving a soil type and wherein the output circuit employs the soil type and the encoding indicating a strength to provide the output indicating the soil moisture as a function of the strength of the encoding of the second radio signal and the soil type.

12. The soil moisture sensor of claim 7 wherein the transponder and second transponder operate independently after receiving the first transmission.

13. The soil moisture sensor of claim 1 wherein the first and second signals are at different frequencies.

14. The soil moisture sensor of claim 1 wherein the predetermined depth is greater than 5 cm.

15. The soil moisture sensor of claim 1 wherein the transponder is free from galvanic connection with the soil.

16. The soil moisture sensor of claim 1 wherein the transponder is free from batteries.
